(19) United States
(12) Patent Application Publication
(10) Pub. No.: US 2021/0369333 A1
(43) Pub. Date: Dec. 2, 2021

(54) MONOLITHIC CERAMIC SURGICAL DEVICE AND METHOD

(71) Applicant: GYRUS ACMI, INC. D/B/A OLYMPUS SURGICAL TECHNOLOGIES AMERICA, Westborough, MA (US)

(72) Inventor: Thomas J. Holman, Princeton, MN (US)

(21) Appl. No.: 17/334,069

(22) Filed: May 28, 2021

Related U.S. Application Data
(60) Provisional application No. 63/032,141, filed on May 29, 2020.

(51) Int. Cl. A61B 18/14; A61B 17/22

(52) U.S. Cl. CPC A61B 18/1447; A61B 2017/2936; A61B 17/22012

(57) ABSTRACT

A medical device and associated methods are disclosed. In one example, the medical device includes an electrosurgical forceps. In selected examples, one or more structural components of the electrosurgical forceps includes a sintered ceramic microstructure. In selected examples other medical devices, including a debrider and a lithotripter, include a sintered ceramic microstructure.

### CLAIM OF PRIORITY

This patent application claims the benefit of priority, under 35 U.S.C. § 119(e), to U.S. Provisional Patent Application Ser. No. 62/032,141, entitled “MONOLITHIC CERAMIC SURGICAL DEVICE AND METHOD,” filed on May 29, 2020, which is hereby incorporated by reference herein in its entirety.

### TECHNICAL FIELD

Embodiments described herein generally relate to medical devices. Specific examples of medical devices include, but are not limited to, forceps, debriders, and lithotripters.

### BACKGROUND

Medical devices for diagnosis and treatment, such as forceps, are often used for medical procedures such as laparoscopic and open surgeries. Forceps can be used to manipulate, engage, grasp, or otherwise affect an anatomical feature, such as a vessel or other tissue of a patient during the procedure. Forceps often include an end effector that is manipulatable from a handle of the forceps. For example, jaws located at a distal end of a forceps can be actuated via elements of the handle between open and closed positions to thereby engage the vessel or other tissue. Forceps can include an extendable and retractable blade that can be extended distally between a pair of jaws to lacerate the tissue. The handle can also be capable of supplying an input energy, such as electromagnetic energy or ultrasound, to the end effector for sealing of a vessel or tissue during a procedure. Improved forceps and other medical devices are desired.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an electrosurgical forceps in accordance with some example embodiments.

FIG. 2A shows a green state ceramic microstructure of a component in an intermediate stage of manufacture of a medical instrument in accordance with some example embodiments.

FIG. 2B shows a sintered ceramic microstructure of a component of a medical instrument in accordance with some example embodiments.

FIG. 3A shows a side view of jaws of an electrosurgical forceps in accordance with some example embodiments.

FIG. 3B shows an isometric view of jaws of an electrosurgical forceps in accordance with some example embodiments.

FIG. 3C shows an electrode located on a jaw of an electrosurgical forceps in accordance with some example embodiments.

FIG. 4A shows one operation of an attachment method using a ceramic component in accordance with some example embodiments.

FIG. 4B shows another operation of an attachment method using a ceramic component in accordance with some example embodiments.

FIG. 4C shows another operation of an attachment method using a ceramic component in accordance with some example embodiments.

FIG. 5 shows a block diagram of a medical device in accordance with some example embodiments.

FIG. 6 shows a portion of a forceps including a jaw region in accordance with some example embodiments.

FIG. 7 shows a portion of a debrider in accordance with some example embodiments.

FIG. 8A shows a lithotripter system in accordance with some example embodiments.

FIG. 8B shows a distal end of a lithotriptor in accordance with some example embodiments.

FIG. 8C shows a distal end of a lithotriptor in accordance with some example embodiments.

FIG. 8D shows a cross section of a lithotripter component in accordance with some example embodiments.

FIG. 9 shows a flow diagram of a method of manufacture of a forceps in accordance with some example embodiments.

### DESCRIPTION OF EMBODIMENTS

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

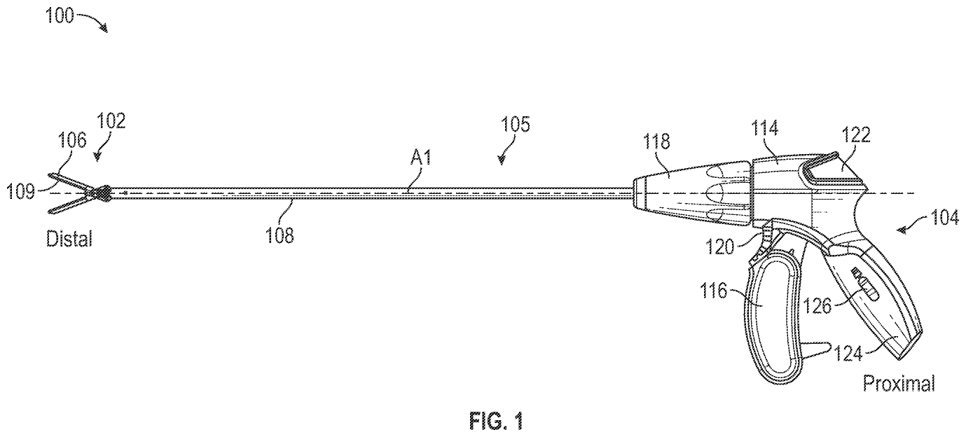

The following disclosure may be used with a number of different types of surgical devices. One example for illustration shown in FIG. 1 is an electrosurgical forceps.

FIG. 1 illustrates a side view of a forceps 100 showing jaws in an open position. The forceps 100 can include an end effector 102, a handpiece 104, and an intermediate portion 105. The end effector 104 can include jaws 106 (including electrodes 109), a shaft 108 is shown located between the end effector 102 and the handpiece 104. In one example, the shaft 108 includes, an inner shaft and an outer shaft, and a blade assembly, although the invention is not so limited. The handpiece 104 can include a housing 114, a lever 116, a rotational actuator 118, a trigger 120, an activation button 122, a handle 124, and a locking mechanism 126. FIG. 1 shows orientation indicators Proximal and Distal and a longitudinal axis A1.

Generally, the handpiece 104 can be located at a proximal end of the forceps 100 and the end effector 102 can be located at the distal end of the forceps 100. The intermediate portion 105 can extend between the handpiece 104 and the end effector 102 to operably couple the handpiece 104 to the end effector 102. Various movements of the end effector 102 can be controlled by one or more actuation systems of the handpiece 104. For example, the end effector 102 can be rotated about the longitudinal axis A1 of the forceps 100. Also, the handpiece can operate the jaws 106, such as by moving the jaws 106 between open and closed position. The handpiece 104 can also be used to operate a cutting blade (not shown) for cutting tissue. The handpiece 104 can also be used to operate the electrode 109 for applying electromagnetic energy to tissue. The end effector 102, or a portion of the end effector 102 can be one or more of: opened, closed, rotated, extended, retracted, and electromagnetically energized.

The housing 114 can be a frame that provides structural support between components of the forceps 100. The housing 114 is shown as housing at least a portion of the actuation systems associated with the handpiece 104 for actuating the end effector 102. However, some or all of the actuation components need not necessarily be contained within the housing 114.

A proximal portion of the trigger 120 can be connected to the blade shaft 112b within the housing 114. A distal portion of the trigger 120 can extend outside of the housing 114 adjacent, and in some examples, nested with the lever 116 in the default or unactuated positions. The activation button 122 can be coupled to the housing 114 and can include or be connected to electronic circuitry within the housing 114. Such circuitry can send or transmit electromagnetic energy through the shaft 108 to the electrodes 109. In some examples, the electronic circuitry may reside outside the housing 114 but may be operably coupled to the housing 114 and the end effector 102.

In operation of the forceps 100, a user can displace the lever 116 proximally to drive the jaws 106 from an open position to a closed position, which can allow the user to clamp down on and compress a tissue. The handpiece 104 can also allow a user to move the rotational actuator 118 to cause the end effector 102 to rotate, such as by rotating the shaft 108, or inner components associated with the shaft 108.

In some examples, with the tissue compressed, a user can depress the activation button 122 to cause electromagnetic energy, or in some examples, ultrasound, to be delivered to one or more components of the end effector 102, such as electrodes 109 and in turn to a tissue. Application of such energy can be used to seal or otherwise affect the tissue. In some examples, the electromagnetic energy can cause tissue to be coagulated, sealed, ablated, or can cause controlled necrosis.

In some examples, the handpiece 104 can enable a user to extend and retract a blade (not shown), which can be attached to a distal end of a blade shaft. In some examples, the blade shaft can extend an entirety of a length between the handle 104 and the end effector 102. The blade can be extended by displacing the trigger 120 proximally and the blade can be retracted by allowing the trigger 120 to return distally to a default position.

The forceps 100 can be used to perform a treatment on a patient, such as a surgical procedure. In one example, a distal portion of the forceps 100, including the jaws 106, can be inserted into a body of a patient, such as through an incision or another anatomical feature of the patient's body. While a proximal portion of the forceps 100, including housing 114 remains outside the incision or another anatomical feature of the body. Actuation of the lever 116 causes the jaws 106 to clamp onto a tissue. The rotational actuator 118 can be rotated via a user input to rotate the jaws 106 for maneuvering the jaws 106 at any time during the procedure. Activation button 122 can be actuated to provide electrical energy to jaws 106 to cauterize or seal the tissue within closed jaws 106. Trigger 120 can be moved to translate a blade assembly distally in order to cut tissue within the jaws 106.

In some examples, the forceps 100, or other medical device, may not include all the features described or may include additional features and functions, and the operations may be performed in any order. The handpiece 104 can be used with a variety of other end effectors to perform other methods.

In one example, one or more of the jaws 106 includes a ceramic microstructure as a structural portion of the jaw. Ceramic materials in surgical tool applications include a number of advantages. One advantage of ceramic materials includes minimal electrical conduction (dielectric behavior) while maintaining desired mechanical properties. With proper material selection, unwanted disadvantages may be avoided.

In one example, the modulus of elasticity of a material substantially governs how the tool feels when compressing a workpiece. For example, when clamping a tissue during a procedure, the jaws of a forceps will flex slightly and provide a clamping force. The amount of flex is determined by the material's modulus of elasticity.

It is desirable, when choosing a material for a forceps or other tool, to provide a tool feel that a user is expecting. If a material has too low of a modulus, the tool may not clamp as effectively. In a sense, it may feel too squishy. If a material has too high of a modulus, the tool may clamp too severely, and unintentional tissue damage may occur. In a sense, the tool may feel too harsh, and not be forgiving enough to accommodate limited control of application force. It is also desirable for a tool to withstand clamping forces, and to not break during use. Because most ceramic materials do not yield before breaking, a tensile strength metric is appropriate to use when comparing to yield strength for metals.

When comparing potential ceramic materials to metals, titanium or stainless steel are good benchmarks. Ranges of yield strength for titanium and titanium alloys are from about 875 MPa to 925 MPa. Ranges of yield strength for stainless steels are from about 200 MPa to 250 MPa. Ranges of modulus of elasticity for titanium and titanium alloys are from about 110 GPa to 120 GPa. Ranges of modulus of elasticity for stainless steel are from about 190 GPa to 200 GPa.

In one example, a ceramic material is selected to feel like a metal component, with the added advantage of being electrically non-conductive. Selected ceramic materials have desired mechanical properties to meet these goals.

In one example, a structural portion of a forceps jaw includes yttria stabilized zirconia. In one example, a structural portion of a forceps jaw includes zirconia toughened alumina. Ranges of modulus of elasticity for yttria stabilized zirconia are from about 200 GPa to 210 GPa. Ranges of modulus of elasticity for zirconia toughened alumina are from about 350 GPa to 370 GPa. Tensile strength for yttria stabilized zirconia is about 500 MPa. Tensile strength for zirconia toughened alumina is about 290 MPa. Although yttria stabilized zirconia and zirconia toughened alumina are used as examples, the invention is not so limited. Other ceramic materials that exhibit dielectric behavior and have elastic moduli similar to metals are also within the scope of the invention.

By choosing a ceramic material with appropriate mechanical properties, a metal component may be replaced with a ceramic component. In one example, this provides a lower cost option of manufacturing. In one example, this provides more options for complex component geometries. In one example, this provides electrical insulation without the need for a separate insulative coating such as a polymer coating.

In one example, a structural component of a medical device is formed from a ceramic material as described in further examples below. Examples of structural components include, but are not limited to, force carrying levers, pivot joints, jaw bodies, impactor heads, cutting blades or other cutting tools, etc.

Ceramic coated components where a structural strength comes from an underlying material, such as a metal, are not considered to be structural components formed from ceramic. In some examples, structural ceramic components, may be combined with metallic components to form a composite component. Composite components may still include a structural portion that is formed from a ceramic as described in examples of the present disclosure.

Uninterrupted portions of sintered ceramic microstructure are defined as monolithic portions. In one example, an entire component, such as a forceps jaw, a debrider cutter, or an impactor includes a monolithic sintered ceramic microstructure. In other examples, only a structural portion of a component, such as a forceps jaw, a debrider cutter, or an impactor includes a monolithic sintered ceramic microstructure.

By forming an entire structural component from a ceramic, the fabrication process for the component is simplified. Only one step of forming is required, instead of a first step of forming, and a separate step of coating as with metal components. Additionally, low cost, highly reliable methods of manufacturing ceramics facilitate fabrication of much higher geometric complexity when compared to metal components. With metal components, frequently sheet metal is used to reduce cost. As such, only flat metal components with bends in the sheet are possible as geometry selections. With sintered ceramic methods as described in the present disclosure, there is no sheet material limitation, and as a result more complex geometries are possible at a low manufacturing cost.

It is important with structural components to select a ceramic material with appropriate material properties. In one example some of the ceramic properties are provided by the material choice. In some examples, the ceramic properties are further provided by the manufacturing process that leads to a desired microstructure.

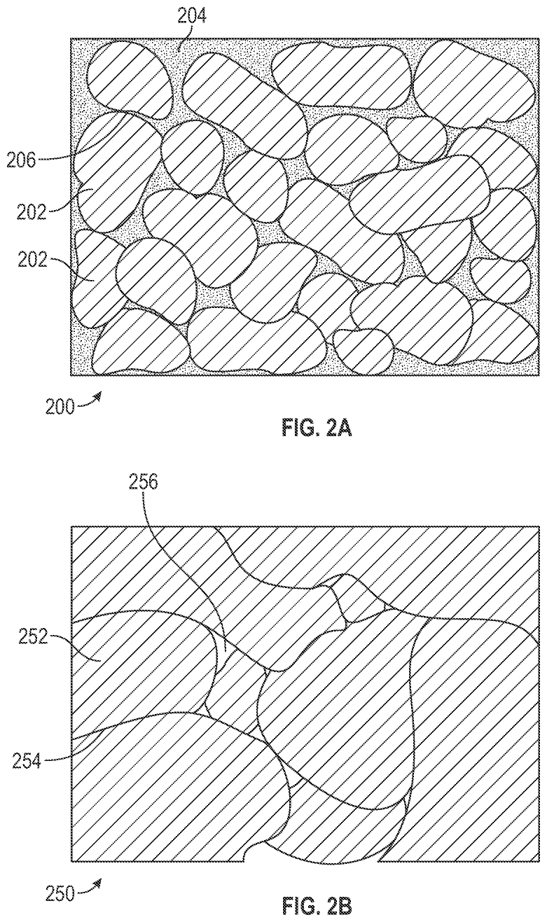

FIG. 2A shows a green state ceramic microstructure 200 according to one example. The green state ceramic microstructure 200 includes a number of ceramic particles 202 and a binder 204. The ceramic particles in the green state contact each other at point contact 206. In one example the binder 204 may include a polymer, or adhesive. In one ceramic manufacturing operation, ceramic particles 202 are combined with binder 204 and pressed into a green state blank. In one example pressing into a green state includes loading ceramic particles 202 and binder 204 into a cylindrical shaped die opening and pressing with a cylinder piston until the ceramic particles 202 and binder 204 are sufficiently densified and held together by the binder. Blanks formed into a green state, as illustrated by the microstructure 200 in FIG. 2A, may be more easily machined, or shaped into a complex geometry prior to sintering, as shown in FIG. 2B.

A green state blank can be any number of shapes. As noted above, in one example, a blank includes a cylinder shape. A cylinder may more easily facilitate a machining operation, such as turning on a lathe while in the green state shown in FIG. 2A. Other machining operations of a green state blank may include milling to form flat sections on a blank. In one example, a computer numerical controlled (CNC) machine may be used to form complex geometries of a component, such as a forceps jaw, a debrider cutter, or an impactor, from the green state blank.

FIG. 2B shows a sintered microstructure 250 after a sintering operation is performed on a green state microstructure as illustrated in FIG. 2A. The sintered microstructure 250 is significantly harder and tougher than the green state microstructure 200 from FIG. 2A. A sintering process burns off the binder 204 from the green state, and material in the ceramic particles 202 migrates and merges between particles 202 to solidify the material.

The ceramic particles 202 with point contacts 206 from FIG. 2A have transformed into grains 252 with long continuous grain boundaries 254. In selected examples some pores 256 remain after sintering. In one example, a selected sintering temperature and time may be selected to control an amount of porosity from pores 256 in a final product. Higher temperatures and longer sintering times may reduce the remaining pores 256 and/or pore size. Advantages of porosity are discussed in more detail in examples below.

Sintering may include elevating a temperature of a green state component in an oven and holding at the temperature for a period of time. One advantage of forming a component from a green state then sintering, includes the ability to easily form complex geometries prior to sintering, while the material is relatively soft. Subsequent sintering then hardens and densities the material with the complex geometry. Machining a sintered or otherwise previously formed ceramic blank may be difficult or impossible due to the high hardness and fracture strength of ceramic materials.

The sintered microstructure 250 of FIG. 2B shows only ceramic grains 252, however the invention is not so limited. Additional components or particles may be included within the microstructure 250 as reinforcement structures or other mechanical property modifiers. In one example, titanium or titanium alloys may be included in the sintered microstructure, for example at grain boundaries, or is pores 256. In one example, tungsten or tungsten alloys may be included. In one example, carbon structures, including, but not limited to, nanotubes, graphite, graphene, etc. may be included in the microstructure 250.

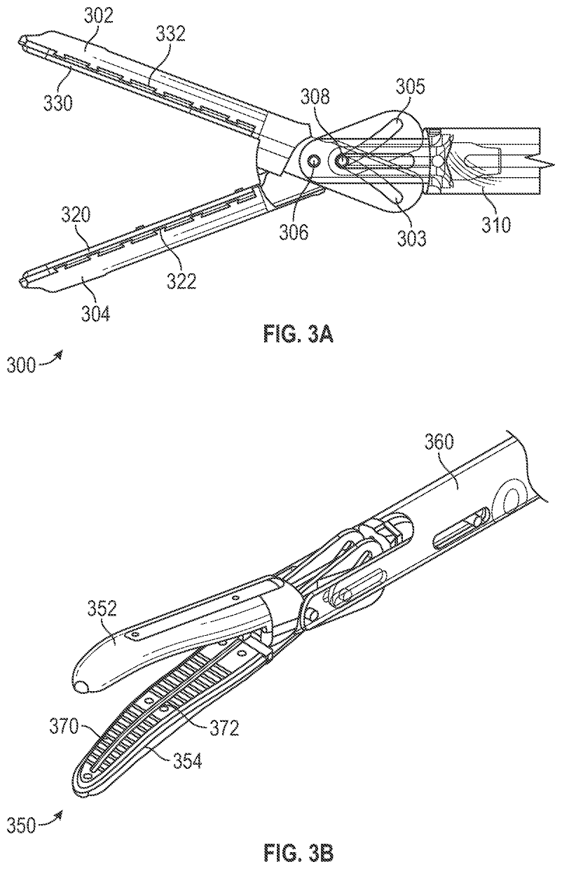

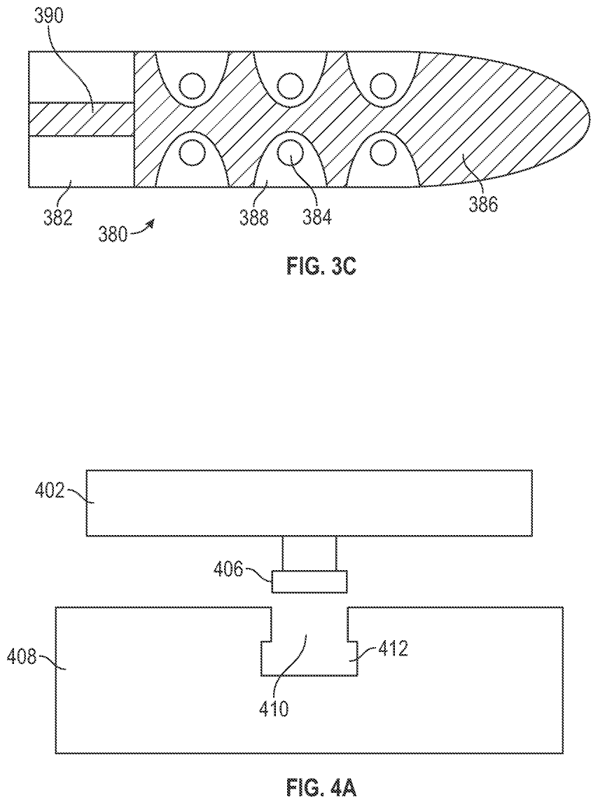

FIG. 3A shows an end effector 300 of a device attached to a distal end of a shaft 310, similar to the forceps 100 shown in FIG. 1. The end effector 300 includes a first forceps jaw 302 and a second forceps jaw 304. In the example shown, the first forceps jaw 302 and the second forceps jaw 304 rotate about a pivot journal 306. In the example of FIG. 3A, both the first forceps jaw 302 and the second forceps jaw 304 are free to rotate, and as such, the set of jaws are dual acting. In other examples, one jaw remains fixed, while the other jaw is allowed to rotate about the pivot journal 306. Only one jaw rotating is defined as single acting.

A cam 308 is shown that travels within cam interfacing slots 303, 305. A first electrode 330 is shown coupled to a first jaw face 332 of the first forceps jaw 302, and a second electrode 320 is shown coupled to a second jaw face 322 of the second forceps jaw 304. In one example one or more of the first and second electrode 330, 320 is a separate component, such as a sheet metal component, that is attached using mechanical, adhesive, or other suitable fastening techniques. In one example one or more of the first and second electrode 330, 320 is deposited or otherwise formed directly over a surface of the sintered ceramic microstructure. Methods of forming include, but are not limited to, plasma spraying, electrodeposition, chemical deposition, sputtering, or other physical vapor deposition.

In one example the first forceps jaw 302 is monolithic and includes a monolithic sintered ceramic microstructure from the first jaw face 332, through the pivot journal 306, and through to the cam interfacing slot 303. In one example, the second forceps jaw 304 likewise includes a monolithic sintered ceramic microstructure. In the example of the first forceps jaw 302, the first jaw face 322 is a structural portion, the pivot journal 306 is structural portion, and the cam interfacing slot 303 is s structural portion. In one example any number of the structural portions may include a monolithic sintered ceramic microstructure as described.

FIG. 3B shows a curved jaw end effector 350. The first jaw 352 and the second jaw 354 are shown. Each jaw 352, 354 includes an electrode 370. Similar to examples described above, in one example an electrode 370 is a separate component, such as a sheet metal component, that is attached using mechanical, adhesive, or other suitable fastening techniques. In one example an electrode 370 is deposited or otherwise formed directly over a surface of the sintered ceramic microstructure. Methods of forming include, but are not limited to, plasma spraying, electrodeposition, chemical deposition, sputtering, or other physical vapor deposition.

One or more protrusions 372 are shown extending above an electrode surface. In operation, it is desirable to bring the electrodes 370 close together, but not in direct contact with one another. In operation, the electrodes 370 are energized when the jaws 352, 354 are closed to provide local heating to tissue clamped between the jaws 352, 354. If the jaws actually touch, a local short circuit will result, and the desired cauterizing of the tissue will not occur.

In one example when sintering manufacturing techniques as described above are used, it is easy to incorporate an integrally formed protrusion 372 on a jaw face. In such an example, the monolithic sintered ceramic microstructure portion includes the protrusion 372.

FIG. 3C shows and example of a jaw 380 that includes protrusions 384. In the example shown, a jaw surface 382 and the protrusions 384 are integrally formed from a green state blank. After sintering, they are monolithic, and include a sintered ceramic microstructure. An electrode 386 is shown extending around the protrusions 384, leaving open spaces 388 in the electrode 386. In the example shown in FIG. 3C, the open spaces 388 extend to an edge of the electrode 380. The protrusions 384 are not completely enclosed laterally by the electrode 386 due to the presence of the open spaces 388. In other examples, the electrode 386 surrounds each protrusion 384. A conductive trace 390 is shown coupled to the electrode 386. In one example the conductive trace 390 is coupled to an energy source such as a battery and/or control circuitry at a proximal end of a device where user controls are located.

One advantage of using a sintered ceramic microstructure for portions of forceps jaws or other end effectors includes the dielectric property of the sintered ceramic microstructure. Because ceramic is a dielectric, there is no need for separate insulating layers such as a polymer coating, to isolate electrical signals or transmitted energy. Metal jaws or other metal end effector components must be coated, or require wires with coated housings to prevent unwanted short circuits.

In one example the conductive trace 390 is deposited or otherwise formed directly over a surface of the sintered ceramic microstructure. In one example one or more of the electrodes is deposited or otherwise formed directly over a surface of the sintered ceramic microstructure. Methods of forming include, but are not limited to, plasma spraying, electrodeposition, chemical deposition, sputtering, or other physical vapor deposition. Depositing an electrode or trace from a vapor, plasma, etc. is easy and inexpensive. When depositing over irregular geometries, it is easy to cover any unusual variations without any undue effort or cost.

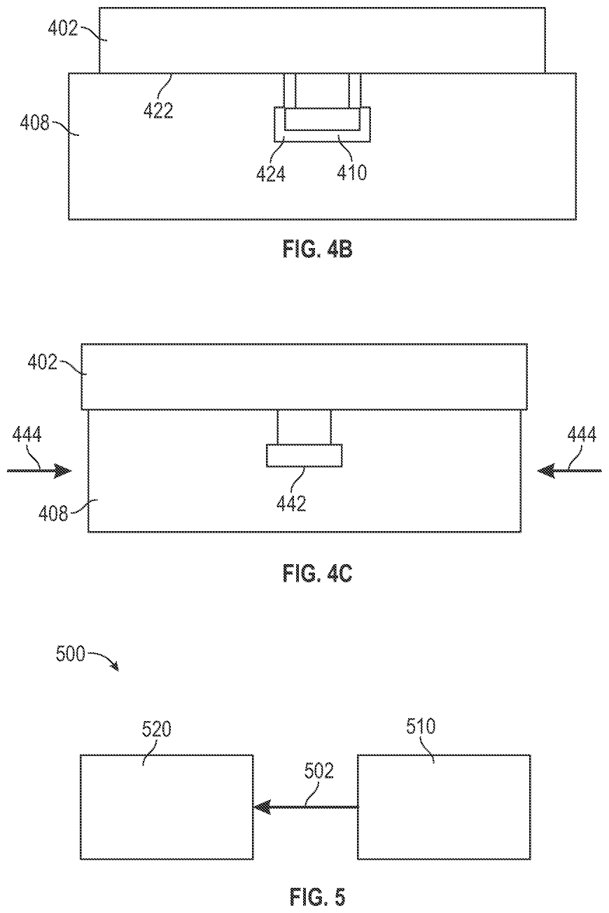

FIGS. 4A-4C illustrate another feature of a sintered ceramic microstructure that is used to join different components in one example. FIG. 4A shows a component 402 having a locking feature 406. A green state ceramic portion 408 is shown with an opening 410. In the example, shown, the opening 410 includes a mating feature 412 that corresponds to the locking feature 406. Because a sintering operation causes a green state component to shrink in a predictable way, a mating feature 412 can be sized to allow the locking feature 406 to enter the opening 410 while in the green state.

FIG. 4B shows how the locking feature 406 can be sized to fit within the opening 410 while the ceramic portion 408 is in the green state. A direct interface 422 is formed between the component 402 and the green state ceramic portion 408. In FIG. 4B, a gap 424 remains between one or more surfaces of the opening 410 and the locking feature 406.

In FIG. 4C, the ceramic portion 408 has undergone sintering, and has undergone shrinking. At least shrinkage along direction 444 provide the locking function. Although homogenous shrinkage is typical, examples are within the scope of the invention where shrinkage along a specific direction is larger than along other directions. In such an example, the ceramic portion 408 is aligned with the desired shrinkage in the desired direction. As shown in FIG. 4C, the locking feature 406 is now locked within the mating feature 412. Because of shrinkage during sintering, gaps 424 are reduced or eliminated, and the locking feature 406 is secured by the sintered ceramic microstructure. Examples of components that may be secured by the procedure described in FIGS. 4A-4C include, but are not limited to, electrodes on jaws, electrical traces, cutters, impactor components, etc.

In one example, when an electro-forceps is used, after clamping a tissue, it may be desired to cauterize the tissue using electrodes as described in examples above. When the tissue is heated by the electrodes, steam may be generated due to the water content of most tissue. In some cases, steam escaping at the electrodes can cause unwanted heat damage to other tissue adjacent to the electrodes on either side. It is desirable to mitigate this issue, or eliminate it entirely. In one example, the steam itself is directed from the electrodes to another location where less damage may occur. In one example, heat from the steam is channeled away using a heat transfer channel, and the remaining steam or water is less damaging due to an amount of heat being removed. Although a forceps is used as an example, the invention is not so limited. Other devices where heat removal is desired may also use configurations described. Examples include, but are not limited to frictional heat dissipation in a rotating cutter or other rotating component.

FIG. 5 shows a block diagram of a device configuration that mitigates or eliminates this heat issue and others. A first region 510 of a device 500 is shown coupled to a second region 520 of the device 500 through a heat transfer channel 502. In one example, an amount of porosity (such as the porosity described in FIG. 2B) provided by the sintered ceramic microstructure can be used as a heat transfer channel 502.

In one example, a sintered ceramic microstructure better facilitates the construction of a heat transfer channel 502 without using porosity. In one example, the heat transfer channel 502 includes a thermal conductive trace that is coupled to the sintered ceramic microstructure. Examples of thermal conductive traces include metallic traces. Metallic traces may be deposited or otherwise attached using methods described above, such as plasma spraying, electrodeposition, chemical deposition, sputtering, or other physical vapor deposition.

In one example, the improved ability to construct complex geometries in a green state, then sinter to form a final component better facilitates construction of a heat transfer channel 502. In one example, the heat transfer channel 502 includes a trench with a metal trace formed within the trench. Such a configuration provides thermal insulation from surrounding tissue or other structures on three sides, with heat conduction being channeled along the metallic trace.

In one example a trench without a metallic trace provides a level of heat transfer. A trench configuration provides a path for moving air or steam to transfer from one location to another where the heat is less damaging. Although a trench is used as an example, other pathways that allow steam or hot gasses to move from one location to another are included in the scope of the invention. In one example, enclosed tubes or other enclosed channels are included. In one example, pathways that are less enclosed than a trench are also included, such as an “L” shaped channel.

Metal traces that are included in a heat transfer channel 502 will be physically distinct, and detectable in a number of configurations. For example, a sputtered or physical vapor deposited metal trace will include a specific grain structure, in contrast to a drawn wire, or other mechanically formed metal conductor. A plasma sprayed metal conductor or a chemical vapor deposited conductor will also include a distinctive physical structure that is detectable in a final product.

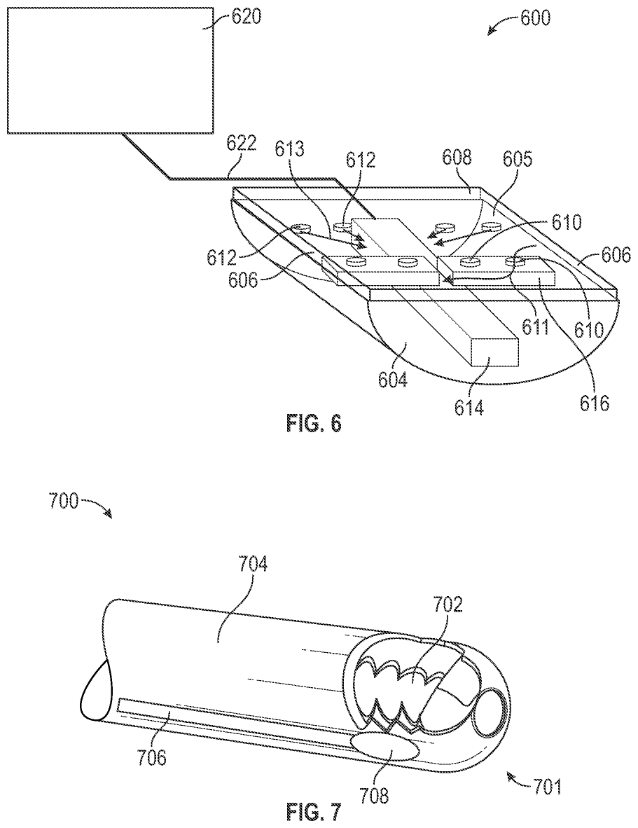

FIG. 6 shows a device 600, including components of an electrosurgical forceps according to one example. The device 600 includes a forceps jaw 604. For illustration purposes, only a cross section of a portion of a forceps jaw 604 is shown in FIG. 6. In one example, the forces jaw 604 includes a sintered ceramic microstructure region. An electrode 608 is coupled to a top surface 605 of the forceps jaw 604.

As discussed above, steam may be generated by heating of tissue using the electrode 608. It is desirable to move steam and/or heat away from edges 606 of the electrode 608. In one example, holes, channels, trenches, or other passages lead from the edges 606 to a location away from the electrode 608. In one example, a heat sink 614 is spaced apart from the electrode 608, and heat and/or stem is directed to the heat sink 614. In operation, the heat sink 614 can safely rise in temperature and hold heat at a safe distance away from the electrode 608 where it will not damage tissue in unwanted locations.

In one example of FIG. 6, first openings 610 in electrode 608 allow steam to re-direct from edges 606 to a heat transfer channel 616. The steam then travels along the heat transfer channel 616 to the heat sink 614, as illustrated by arrow 611. In one example, the heat transfer channel 616 includes a thermally conductive material coupled to the sintered ceramic microstructure of the forceps jaw 604. In one example, a thermal conduction coefficient of the thermally conductive material is higher than the sintered ceramic microstructure to facilitate thermal conduction. Examples of the thermally conductive material include but are not limited to metals and metal alloys. In one example, using a thermally conductive material in the heat transfer channel 616, heat is transferred through the thermally conductive material, but not necessarily steam, which may be physically blocked by the thermally conductive material. Cooling of any steam generated is accomplished by transfer of the heat from the steam at edges 606.

In another example, an open passage, such as a trench, channel, or other directed open space is used in the heat transfer channel 616. In such an example, steam itself may be allowed to physically escape through the passage from the edges 606 to the heat sink 614. In one example a combination of a thermally conductive material and an open passage enhances the transfer of both steam and heat from the steam.

Also shown in FIG. 6 are second openings 612 that allow steam to pass from edges 606 through the electrode 608 into a portion of the forceps jaw 604 that includes at least some amount of porosity. In one example, the sintered ceramic microstructure of the portion of the forceps jaw 604 is located adjacent to the electrode 608, such that the porosity permits escape of steam from edges 606. In the example of FIG. 6, steam is directed through porosity in the sintered ceramic microstructure as shown by arrows 613. In one example, the steam is directed to the heat sink 614. In other examples, steam is directed to a location away from the electrode 608, but not necessarily to a heat sink 614.

In fabrication of the forceps jaw 604, an amount of porosity and a structure of the porosity can be controlled by varying processing conditions. For example shorter heating times may start a sintering process that joins ceramic particles from the green state at contact points, but leave pores behind. Longer heating times may further complete a sintering process and reduce porosity. In other examples, varying starting ceramic particle size in the green state may control sizes of pores. Varying porosity provides a control of speed and amount of steam transfer as described above.

Although openings 610, 612 are shown as round holes, the invention is not so limited. Any passage or channel that allows steam and/or heat to move away from a portion, such as edges 606 of the electrode 608 are within the scope of the invention. For example, cut outs in sides of the electrode 608 may also allow passage of heat and/or steam from edges 606. Additionally, although heat transfer channels 616 are shown in block diagram form as rectangles, any geometry that allows passage of heat and/or steam from edges 606 is within the scope of the invention. Also, although a heat sink 614 is shown, to collect heat in FIG. 6, other selected example devices merely channel heat and/or steam away from edges 606 without a specific structure such as a heat sink 614 to hold heat in any one location.

FIG. 6 also shows an optional heat pipe 620 coupled to the heat sink 614 through connection 622. A heat pipe 620, or other cooling structure such as cooling fins, a Peltier device, etc. may be used to provide further cooling to examples described. In one example, the heat pipe 620 further dissipates heat by evaporating an enclosed medium such as water within the heat pipe, and cooling the evaporated water at a distal location. FIG. 6 illustrates the heat pipe 620 in a block diagram form. One of ordinary skill in the art, having the benefit of the present disclosure, will recognize that portions of the heat pipe 620 may be directly coupled to the heat transfer channel 616, or be coupled to an intermediate heat sink 614.

Other devices also benefit from a sintered ceramic microstructure as described in examples above. FIG. 7 shows a debrider 700. The debrider includes an end effector 701 coupled to an end of a shaft 704. In one example, the end effector 701 includes a cyclic blade and a corresponding blade adjacent to an edge of the cyclic blade. In FIG. 7, a stationary blade 702 is visible, and a cyclic blade (not shown) rotates or cycles within the stationary blade 702. In one example, one or more components of the end effector 701 includes a sintered ceramic microstructure. For example, either one, or both of the blades may include a sintered ceramic microstructure. In one example, all or part of the shaft 704 includes a sintered ceramic microstructure. Advantages over metal include, but are not limited to, simplified manufacturing of complex geometries, wear resistance for frictional components such as rotating cutters, and electrical resistivity.

FIG. 7 further shows an electrical trace 706. In one example, an electrical trace 706 may be used to channel heat away from the end effector 701, such as heat generated by friction of the cyclic blade. In one example, an electrode 708 is coupled to the electrical trace 706. Electrode 708 may be part of a sensor, and provide a number of functions, including, but not limited to, heat sensing, sensing of body chemistry, sensing applied drug chemistry, receiving or transmitting electrical signals, etc. The electrical resistivity advantage of a sintered ceramic microstructure facilitates easy deposition of electrical traces 706 on a surface of the sintered ceramic microstructure. Additionally, the electrical trace 706 may be formed within complex geometry, such as a trench, that provides increased protection of the electrical trace 706, while still being electrically isolated. In selected examples, the sintered ceramic microstructure is included on a structural portion of the debrider 700. In selected examples, the sintered ceramic microstructure is included on a non-structural portion of the debrider 700.

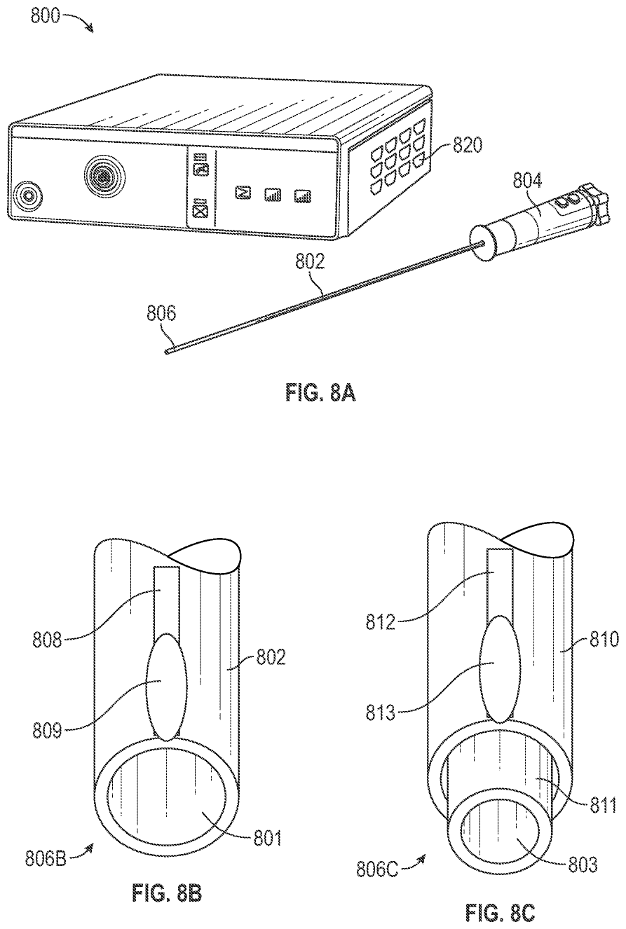

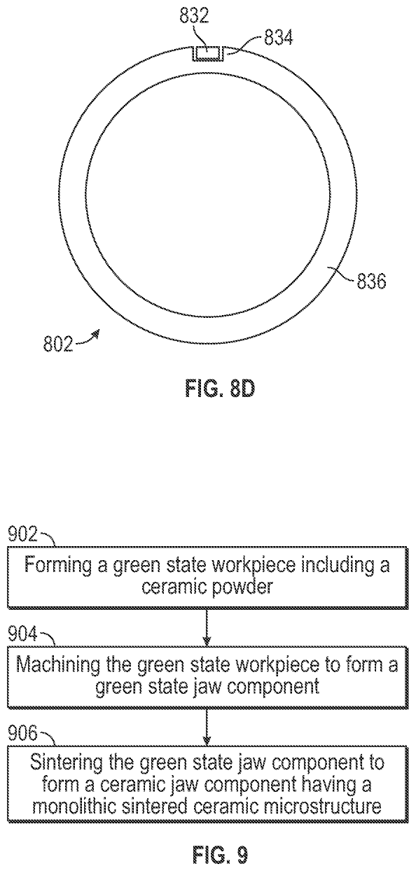

FIG. 8A shows another device that benefits from a sintered ceramic microstructure as described in examples above. FIG. 8A shows a lithotripter 800 according to one example. In FIG. 8A, a hollow shaft 802 extends from a handpiece 804. A controller 820 is used in conjunction with the shaft 802 and handpiece 804 through connecting lines not shown. In one example, at least a portion of a distal end 806 of the shaft 802 includes a monolithic sintered ceramic microstructure. Similar to the example of the debrider of FIG. 7, advantages over metal include, but are not limited to, simplified manufacturing of complex geometries, and electrical resistivity. Cyclic components in selected examples of lithotripters also benefit from improved wear reduction and friction reduction over metal.

FIG. 8B shows one example of an impact surface at a distal end 806B of a shaft. In operation, the shaft 802 is vibrated at a selected frequency, and obstructions, such as kidney stones, are impacted using the distal end 806B. Broken portions are then removed through central opening 801. In one example, the distal portion 806B of the shaft 802 shown in FIG. 8B includes a monolithic sintered ceramic microstructure. In one example, at least the distal portion 806B is structural, in contrast to a ceramic coating over a metal, where the metal provides structure.

FIG. 8B further shows an electrical trace 808. As in examples above, an electrical trace 808 may be used to channel heat away from the distal portion 806B. In one example, an electrode 809 is coupled to the electrical trace 808. Electrode 809 may be part of a sensor, and provide a number of functions, including, but not limited to, heat sensing, sensing of body chemistry, sensing applied drug chemistry, receiving or transmitting electrical signals, etc.

FIG. 8C shows another example of an impact surface at a distal end 806C of a shaft. The distal end 806C includes an outer tube 810 and an inner tube 811. In operation, the inner tube 811 is cycled back and forth within the outer tube 810. Obstructions, such as kidney stones, are impacted using the distal end 806B. Broken portions are then removed through central opening 803. In one example, one or more components of the distal portion 806C shown in FIG. 8C includes a monolithic sintered ceramic microstructure. For example, a portion of one or both tubes 810, 811 may include a monolithic sintered ceramic microstructure. In one example, at least the distal portion 806C is structural, in contrast to a ceramic coating over a metal, where the metal provides structure.

Similar to FIG. 8B, in one example, the distal end 806C further shows an electrical trace 812. As in examples above, an electrical trace 812 may be used to channel heat away from the distal portion 806C. In one example, an electrode 813 is coupled to the electrical trace 812. Electrode 813 may be part of a sensor, and provide a number of functions, including, but not limited to, heat sensing, sensing of body chemistry, sensing applied drug chemistry, receiving or transmitting electrical signals, etc.

FIG. 8D shows a cross section of a portion of shaft 802 according to one example. In FIG. 8D, an electrical trace 832, similar to electrical trace 808 or 812 described above, is recessed at least partially within a trench 834. The trench 834 is formed within a sidewall 836 of the shaft 802. As described in examples above, one advantage of a sintered ceramic microstructure includes the ability to manufacture complex geometries such as trench 834. Further, the electrical resistivity of ceramic provides electrical isolation of the trace 832 on three sides as illustrated in FIG. 8D.

FIG. 9 shows one example flow diagram of a method of making a forceps. In operation 902, a green state workpiece is formed including a ceramic powder. In operation 904, the green state workpiece is machined to form a green state jaw component. In operation 906, the green state jaw component is sintered to form a ceramic jaw component having a monolithic sintered ceramic microstructure. Although a forceps jaw is described in the manufacturing steps of FIG. 9, other components for other devices may be similarly manufactured. For example, a debrider component or a lithotripter component may be manufactured in similar operations.

To better illustrate the method and apparatuses disclosed herein, a non-limiting list of embodiments is provided here:

Example 1 includes a forceps jaw. The forceps jaw includes a jaw contact surface and an electrode coupled to the jaw contact surface, wherein a monolithic sintered ceramic microstructure is a structural portion of the jaw.

Example 2 includes the forceps jaw of example 1, wherein the monolithic sintered ceramic microstructure includes yttria stabilized zirconia.

Example 3 includes the forceps jaw of any one of examples 1-2, wherein the monolithic sintered ceramic microstructure includes zirconia toughened alumina.

Example 4 includes the forceps jaw of any one of examples 1-3, wherein the structural portion of the forceps jaw includes a pivot journal.

Example 5 includes the forceps jaw of any one of examples 1-4, wherein the structural portion of the forceps jaw includes a cam interfacing slot.

Example 6 includes the forceps jaw of any one of examples 1-5, wherein the electrode includes a locking feature that is secured by a sintered ceramic feature.

Example 7 includes the forceps jaw of any one of examples 1-6, further including an electrical trace coupled to the electrode, the electrical trace attached to a surface of the monolithic sintered ceramic microstructure of the forceps jaw.

Example 8 includes the forceps jaw of any one of examples 1-7, further including at least one protrusion coupled to the jaw contact surface, wherein the at least one protrusion is sized or arranged to extend above an electrode surface to keep the electrode from contacting an opposing electrode when the forceps jaw is in a closed position.

Example 9 includes the forceps jaw of any one of examples 1-8, wherein the at least one protrusion is integrally formed from the monolithic sintered ceramic microstructure.

Example 10 includes a debrider. The debrider includes a number of end effector components located at an end of a shaft. The end effector components include a cyclic blade, and a corresponding blade adjacent to an edge of the cyclic blade, wherein one or more of the end effector components includes a monolithic sintered ceramic microstructure.

Example 11 includes the debrider of example 10, further including an electrical trace coupled a surface of the monolithic sintered ceramic microstructure.

Example 12 includes the debrider of any one of examples 10-11, wherein the number of end effector components further includes a cauterizing electrode, and wherein the electrical trace is coupled to the cauterizing electrode.

Example 13 includes the debrider of any one of examples 10-12, wherein the electrical trace is recessed within a trench in the monolithic sintered ceramic microstructure.

Example 14 includes a lithotripter. The lithotripter includes a hollow shaft extending from a handpiece, and an impact surface located at a distal end of the hollow shaft, wherein at least a portion of the distal end of the shaft includes a monolithic sintered ceramic microstructure.

Example 15 includes the lithotripter of example 14, further including an electrical trace coupled to a surface of the monolithic sintered ceramic microstructure.

Example 16 includes the lithotripter of any one of examples 14-15, wherein the electrical trace is recessed within a trench in the monolithic sintered ceramic microstructure.

Example 17 includes the lithotripter of any one of examples 14-16, wherein the impact surface includes a monolithic sintered ceramic microstructure.

Example 18 includes a forceps. The forceps includes jaws located at an end of a shaft, a jaw actuator routed along the shaft and coupled to one or more of the jaws, and a pair of electrodes coupled to opposing surfaces of jaws wherein at least one of the jaws includes a sintered ceramic microstructure region. The forceps includes a heat transfer channel in the sintered ceramic microstructure region, to preferentially direct heat away from a first electrode of the pair of electrodes when in operation.

Example 19 includes the forceps of example 18, wherein only one of the jaws is movable with respect to the shaft in response to the jaw actuator.

Example 20 includes the forceps of any one of examples 18-19, wherein two jaws are both movable with respect to the shaft in response to the jaw actuator.

Example 21 includes the forceps of any one of examples 18-20, wherein the heat transfer channel includes a thermally conductive material coupled to the sintered ceramic microstructure, wherein a thermal conduction coefficient of the thermally conductive material is higher than the sintered ceramic microstructure.

Example 22 includes the forceps of any one of examples 18-21, wherein the heat transfer channel includes an open space at least partially within walls to direct steam from a first electrode of the pair of electrodes when in operation.

Example 23 includes the forceps of any one of examples 18-22, further including a heat sink located apart from the pair of electrodes, wherein the heat transfer channel is routed between the first electrode and the heat sink.

Example 24 includes the forceps of any one of examples 18-23, further including a heat pipe located apart from the pair of electrodes, wherein the heat transfer channel is routed between the first electrode and the heat pipe.

Example 25 includes a forceps. The forceps includes jaws located at an end of a shaft, a jaw actuator routed along the shaft and coupled to one or more of the jaws, and a pair of electrodes coupled to opposing surfaces of jaws wherein at least one of the jaws includes a sintered ceramic microstructure region having a porosity, and wherein the sintered ceramic microstructure region is located adjacent to a first electrode of the pair of electrodes, such that the porosity permits escape of steam from near the first electrode of the pair of electrodes when in operation.

Example 26 includes the forceps of example 25, further including a heat sink located apart from the pair of electrodes, wherein the porosity directs steam between the first electrode and the heat sink when in operation.

Example 27 includes the forceps of any one of examples 25-26, further including a heat pipe located apart from the pair of electrodes, wherein the porosity directs steam between the first electrode and the heat pipe when in operation.

Example 28 includes a method of making a forceps. The method includes forming a green state workpiece including a ceramic powder, machining the green state workpiece to form a green state jaw component, and sintering the green state jaw component to form a ceramic jaw component having a monolithic sintered ceramic microstructure.

Example 29 includes the method of example 28, further including attaching an electrode to a grasping surface of the ceramic jaw component.

Example 30 includes the method of any one of examples 28-29, wherein attaching an electrode includes plasma spraying a metal onto the ceramic jaw component.

Example 31 includes the method of any one of examples 28-30, wherein attaching an electrode includes sputtering a metal onto the ceramic jaw component.

Example 32 includes the method of any one of examples 28-31, wherein attaching an electrode includes inserting an electrode feature of a separately formed electrode into a cavity within the green state jaw component and shrinking the cavity over the electrode feature as a result of sintering.

Example 33 includes the method of any one of examples 28-32, further including attaching a conductive trace onto the ceramic jaw component and coupling the conductive trace to the electrode.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. A forceps jaw, comprising:
a jaw contact surface;
an electrode coupled to the jaw contact surface; and
wherein a monolithic sintered ceramic microstructure is a structural portion of the j aw.

2. The forceps jaw of claim 1, wherein the monolithic sintered ceramic microstructure includes yttria stabilized zirconia.

3. The forceps jaw of claim 1, wherein the monolithic sintered ceramic microstructure includes zirconia toughened alumina.

4. The forceps jaw of claim 1, wherein the structural portion of the forceps jaw includes a pivot journal.

5. The forceps jaw of claim 1, wherein the structural portion of the forceps jaw includes a cam interfacing slot.

6. The forceps jaw of claim 1, wherein the electrode includes a locking feature that is secured by a sintered ceramic feature.

7. The forceps jaw of claim 1, further including an electrical trace coupled to the electrode, the electrical trace attached to a surface of the monolithic sintered ceramic microstructure of the forceps jaw.

8. The forceps jaw of claim 1, further including at least one protrusion coupled to the jaw contact surface, wherein the at least one protrusion is sized or arranged to extend above an electrode surface to keep the electrode from contacting an opposing electrode when the forceps jaw is in a closed position.

9. The forceps jaw of claim 8, wherein the at least one protrusion is integrally formed from the monolithic sintered ceramic microstructure.

10. A debrider, comprising
a number of end effector components located at an end of a shaft, the end effector components including:
a cyclic blade; and
a corresponding blade adjacent to an edge of the cyclic blade;
wherein one or more of the end effector components includes a monolithic sintered ceramic microstructure.

11. The debrider of claim 10, further including an electrical trace coupled a surface of the monolithic sintered ceramic microstructure.

12. The debrider of claim 10, wherein the number of end effector components further includes a cauterizing electrode, and wherein the electrical trace is coupled to the cauterizing electrode.

13. The debrider of claim 11, wherein the electrical trace is recessed within a trench in the monolithic sintered ceramic microstructure.

14. A lithotriptor, comprising
a hollow shaft extending from a handpiece;
an impact surface located at a distal end of the hollow shaft;
wherein at least a portion of the distal end of the shaft includes a monolithic sintered ceramic microstructure.

15. The lithotriptor of claim 14, further including an electrical trace coupled to a surface of the monolithic sintered ceramic microstructure.

16. The lithotriptor of claim 15, wherein the electrical trace is recessed within a trench in the monolithic sintered ceramic microstructure.

17. The lithotriptor of claim 14, wherein the impact surface includes a monolithic sintered ceramic microstructure.

18. A forceps, comprising:
jaws located at an end of a shaft;
a jaw actuator routed along the shaft and coupled to one or more of the jaws;
a pair of electrodes coupled to opposing surfaces of jaws;
wherein at least one of the jaws includes a sintered ceramic microstructure region; and
a heat transfer channel in the sintered ceramic microstructure region, to preferentially direct heat away from a first electrode of the pair of electrodes when in operation.

19. The forceps of claim 18, wherein only one of the jaws is movable with respect to the shaft in response to the jaw actuator.

20. The forceps of claim 18, wherein two jaws are both movable with respect to the shaft in response to the jaw actuator.

21. The forceps of claim 18, wherein the heat transfer channel includes a thermally conductive material coupled to the sintered ceramic microstructure, wherein a thermal conduction coefficient of the thermally conductive material is higher than the sintered ceramic microstructure.

22. The forceps of claim 18, wherein the heat transfer channel includes an open space at least partially within walls to direct steam from a first electrode of the pair of electrodes when in operation.

23. The forceps of claim 18, further including a heat sink located apart from the pair of electrodes, wherein the heat transfer channel is routed between the first electrode and the heat sink.

24. The forceps of claim 18, further including a heat pipe located apart from the pair of electrodes, wherein the heat transfer channel is routed between the first electrode and the heat pipe.

25. A forceps, comprising:
jaws located at an end of a shaft;
a jaw actuator routed along the shaft and coupled to one or more of the jaws;
a pair of electrodes coupled to opposing surfaces of jaws;
wherein at least one of the jaws includes a sintered ceramic microstructure region having a porosity; and
wherein the sintered ceramic microstructure region is located adjacent to a first electrode of the pair of electrodes, such that the porosity permits escape of steam from near the first electrode of the pair of electrodes when in operation.

26. The forceps of claim 25, further including a heat sink located apart from the pair of electrodes, wherein the porosity directs steam between the first electrode and the heat sink when in operation.

27. The forceps of claim 25, further including a heat pipe located apart from the pair of electrodes, wherein the porosity directs steam between the first electrode and the heat pipe when in operation.

28. A method of making a forceps, comprising:
forming a green state workpiece including a ceramic powder;
machining the green state workpiece to form a green state jaw component; and
sintering the green state jaw component to form a ceramic jaw component having a monolithic sintered ceramic microstructure.

29. The method of claim 28, further including attaching an electrode to a grasping surface of the ceramic jaw component.

30. The method of claim 29, wherein attaching an electrode includes plasma spraying a metal onto the ceramic jaw component.

31. The method of claim 29, wherein attaching an electrode includes sputtering a metal onto the ceramic jaw component.

32. The method of claim 29, wherein attaching an electrode includes inserting an electrode feature of a separately formed electrode into a cavity within the green state jaw component and shrinking the cavity over the electrode feature as a result of sintering.

33. The method of claim 29, further including attaching a conductive trace onto the ceramic jaw component and coupling the conductive trace to the electrode.
